(19) United States
(12) Patent Application Publication
(10) Pub. No.: US 2026/0000513 A1
(43) Pub. Date: Jan. 1, 2026

(54) PENILE IMPLANT WITH MULTIPLE CONFIGURATIONS

(71) Applicant: Boston Scientific Scimed, Inc., Maple Grove, MN (US)

(72) Inventors: Jonathan Melchor, Providence, RI (US); Stephanie A. George, St. Louis Park, MN (US)

(21) Appl. No.: 19/249,550

(22) Filed: Jun. 25, 2025

Related U.S. Application Data
(60) Provisional application No. 63/666,375, filed on Jul. 1, 2024.

(51) Int. Cl. A61F 2/26; A61F 2/00; A61F 2/48

(52) U.S. Cl. CPC A61F 2/26; A61F 2/0077; A61F 2/48

(57) ABSTRACT

According to an aspect, a penile implant includes an articulating portion, a control portion, and an elongate member. The elongate member extends from the control portion to the articulating portion. In some aspects, the articulating portion includes a plurality of articulating members and a plurality of locking members.

### CROSS-REFERENCE TO RELATED APPLICATION

This application claims priority to U.S. Provisional Patent Application No. 63/666,375, filed on Jul. 1, 2024, entitled “PENILE IMPLANT WITH MULTIPLE CONFIGURATIONS”, the disclosure of which is incorporated by reference herein in its entirety.

### TECHNICAL FIELD

This disclosure relates generally to bodily implants and more specifically to bodily implants, such as penile prostheses, that may be placed in multiple configurations.

### BACKGROUND

One treatment for male erectile dysfunction is the implantation of a penile prosthesis that may be placed in multiple configurations. For example, some existing penile prostheses may be implanted in or near the penis of the patient and may be disposed in a first configuration to place the penis in a flaccid-like state and may be disposed in a second configuration to place the penis in an erect-like state. Some existing implants include complex activation mechanisms to change the configurations of the implants and may require a large amount of effort to change or convert the implant from one configuration to another configuration.

Accordingly, it would be useful to provide a bodily implant, such as a penile prosthesis, that may efficiently be moved from one configuration to another configuration. For example, it would be useful to provide a bodily implant, such as a penile prosthesis, that may be placed in a rigid configuration and a flexible configuration.

### SUMMARY

According to an aspect, a penile implant includes an articulating portion, a control portion, and an elongate member. The elongate member extends from the control portion to the articulating portion. In some aspects, the articulating portion includes a plurality of articulating members and a plurality of locking members.

In some implementations, the articulating portion includes a first articulating member, a second articulating member, and a locking member disposed between the first articulating member and the second articulating member. In some implementations, the articulating portion includes a first locking member, a second locking member, and an articulating member disposed between the first locking member and the second locking member. In some implementations, the articulating portion includes an articulating member and a locking member, the articulating member defines a cavity, at least a portion of the locking member being configured to be disposed within the cavity defined by the articulating member. In some implementations, the articulating portion includes an articulating member and a locking member, the articulating member defines a cavity, at least a portion of the locking member being configured to be disposed within the cavity defined by the articulating member, the articulating member includes an engagement portion that is configured to engage an engagement portion of the locking member.

In some implementations, the articulating portion includes an articulating member and a locking member, the articulating member defines a cavity, at least a portion of the locking member being configured to be disposed within the cavity defined by the articulating member, the articulating member includes a projection disposed within the cavity, the projection of the articulating member is configured to engage a groove of the locking member. In some implementations, the articulating portion includes a first articulating member, a second articulating member, and a locking member, the first articulating member defines a cavity, at least a portion of the locking member being configured to be disposed within the cavity defined by the first articulating member, the first articulating member includes a projection disposed within the cavity, the projection of the first articulating member is configured to engage a groove of the locking member, the second articulating member defines a cavity, at least a portion of the locking member being configured to be disposed within the cavity defined by the second articulating member, the second articulating member includes a projection disposed within the cavity, the projection of the second articulating member is configured to engage a groove of the locking member.

In some implementations, the control portion includes a plunger member and a cam member. In some implementations, the control portion includes a plunger member and a cam member, the plunger defines a lumen, the cam member defines a lumen. In some implementations, the control portion includes a plunger member, a cam member, and a housing member. In some implementations, the control portion includes a plunger member and a cam member, the plunger member having an activation surface.

In some implementations, the control portion includes a plunger member and a cam member, the plunger member having a first activation surface disposed on a first side portion of the plunger member, the plunger member having a second activation surface disposed on a second side portion of the plunger member, the first side portion being opposite the second side portion of the plunger member. In some implementations, the control portion includes a plunger member, a cam member, housing member, and a bias member. In some implementations, the control portion includes a housing member and a lock member configured to extend through an opening defined by the housing member.

In another aspect, a penile implant includes an articulating portion having a housing member defining a cavity, a plunger member, and a cam member, at least a portion of the plunger member being disposed within the cavity of the housing member, at least a portion of the cam member being disposed within the cavity of the housing member; a control portion; and an elongate member extending from the control portion to the articulating portion.

In some implementations, the articulating portion includes a plurality of articulating members and a plurality of locking members. In some implementations, the articulating portion includes a first articulating member, a second articulating member, and a locking member disposed between the first articulating member and the second articulating member.

In yet another aspect, a method of using a penile implant includes actuating a lock member; and engaging an activation surface of a plunger member of the penile implant to convert the penile implant from a linear configuration to a non-linear configuration.

In some implementations, the activation surface of the plunger member is a first activation surface of the plunger member, the method further includes engaging a second activation surface of the plunger member.

### BRIEF DESCRIPTION OF THE DRAWINGS

FIG. 1 schematically illustrates an implant, such as a penile prosthesis, according to an implementation.

FIG. 2 is a see-through side view of a penile prosthesis according to an implementation in a first configuration.

FIG. 3 is a side view of the penile prosthesis of FIG. 2 in a second configuration.

FIG. 4 is a perspective view of an articulating member of the penile prosthesis of FIG. 2.

FIG. 5 is a perspective view of a locking member of the penile prosthesis of FIG. 2.

FIG. 6 is an exploded view of an articulating portion of the penile prosthesis of FIG. 2.

FIG. 7A is an end view of an articulating member and a locking member.

FIGS. 7B and 7C are perspective views of a portion of an articulating portion of a penile prosthesis.

FIG. 8 is a perspective view of a plunger member of the penile prosthesis of FIG. 2.

FIG. 9 is a perspective view of a cam member of the penile prosthesis of FIG. 2.

FIG. 10 is a perspective view of a housing member of the penile prosthesis of FIG. 2.

FIG. 11 is a perspective view of the plunger member of FIG. 8 and the cam member of FIG. 9 in a collapsed configuration.

FIG. 12 is a perspective view of the plunger member of FIG. 8, the cam member of FIG. 9, and the housing member of FIG. 10 in a collapsed configuration.

FIG. 13 is a perspective view of the plunger member of FIG. 8 and the cam member of FIG. 9 in an extended configuration.

FIG. 14 is a perspective view of the plunger member of FIG. 8, the cam member of FIG. 9, and the housing member of FIG. 10 in an extended configuration.

FIGS. 15-18 are perspective views of the plunger member of FIG. 8 and the cam member of FIG. 9 transitioning from an extended configuration to a collapsed configuration.

FIGS. 19-22 are perspective views of the plunger member of FIG. 8 and the cam member of FIG. 9 transitioning from a collapsed configuration to an extended configuration.

FIGS. 23-26 illustrate implants in accordance with implementations.

FIG. 27 illustrates a portion of an implant in accordance with an implementation.

FIGS. 28-34 illustrate portions of implants in accordance with implementations.

FIG. 35 illustrates end extenders of an implant according to implementations.

FIG. 36 is a flow chart of a method according to an implementation.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to medical devices or implants such as penile prostheses or other bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human male, a human female, or any other mammal.

The embodiments discussed herein may improve the performance of a penile prosthesis. For example, the penile prosthesis may have improved usability or functionality. Specifically, in some embodiments, the implant or penile prosthesis may require a small amount of effort to convert the implant from a first configuration to a second configuration. In some embodiments, the implant or penile prosthesis may be able to be placed in linear and curved configurations.

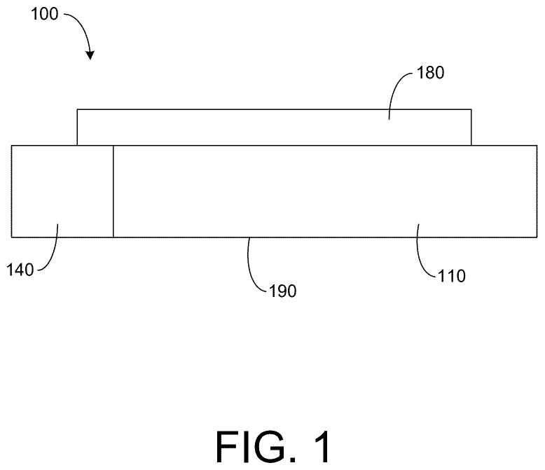

FIG. 1 schematically illustrates prosthesis or an implant 100 according to an aspect. The implant 100 includes an articulating portion 110 and a control portion 140. The implant 100 also includes an elongate member 180 that extends from the control portion 140 to the articulating portion 110. In some implementations, the implant 100 may be a penile implant and the implant 100 may be placed within a penis of a patient. Specifically, in some implementations, the implant 100 may be implanted within the corpus cavernosae of the patient. In other implementations, the implant may be placed in other locations within the body of the patient.

In some implementations, more than one implant 100 may be placed within the body of the patient. For example, in some implementations, two implants 100 may be placed within the body of the patient. Specifically, in some implementations, two implants 100 may be placed within a penis of the patient. The implants may be placed within the penis of the patient in substantially parallel or a side-by-side arrangement. In other implementations, only one implant 100 is disposed or implanted within the body of the patient.

The implant 100 may be placed in a variety of configurations. As will be described in more detail below, the implant 100 may be placed in a curved configuration or a linear or substantially linear configuration. Accordingly, in some implementations, the implant 100 may be placed within a penis of the patient and the patient may selectively place the implant 100 in (1) a straight or linear configuration to achieve or simulate an erect penis or (2) a curved or non-linear configuration to achieve or simulate a flaccid penis.

The articulating portion 110 is configured to be placed in a curved configuration and a linear or substantially linear configuration. In some implementations, the linear or substantially linear configuration has less curve or curvature than when the articulating portion is in its curved configuration. In some implementations, the articulating portion includes a plurality of articulating members (such as two or more than two) that is configured to move, pivot, or rotate with respect to each other. In some implementations, the plurality of articulating members is configured to move with respect to each other such that the articulating portion 110 may be placed in a curved configuration and a linear or substantially linear configuration.

In some implementations, the articulating portion 110 also includes a plurality of locking members (such as two or more than two). The locking members are configured to fix or lock the articulating members in place with respect to each other. For example, in some implementations, the locking members are configured to engage the articulating members to fix or lock the articulating members in place to place the articulating portion 110 in a linear or substantially linear configuration.

The control portion 140 is configured to be engaged by the user to place the implant 100 (or the articulating portion 110) in a curved configuration or a linear or substantially linear configuration.

In the illustrated implementations, the implant 100 includes an elongate member 180 that extends from the control portion 140 to the articulating portion 110. In some implementations, the elongate member 180 is configured to move from a first position with respect to the articulating portion 110 to a second position with respect to the articulating portion 110. The movement of the elongate member 180 may cause the elongate member 180 (and the implant 100) to be converted from one configuration (such as a curved configuration) to another configuration (such as a linear or substantially linear configuration). In some implementations, the elongate member 180 is a wire or cable of a fixed length that is configured to flex or bend.

In some implementations, the implant 100 includes a sheath or cover member 190. In some implementations, the cover member extends around or surrounds the outer surface of the implant 100. For example, in some implementations, the cover member defines a cavity and the control portion 140 and the articulating portion 110 are disposed within the cavity defined by the cover member. In some implementations, the cover member is coupled to a portion of the implant 100. In some implementations, the cover member is flexible and/or configured to stretch. In such implementations, the cover member flexes and/or stretches when the implant 100 is moved from one of its configurations to another of its configurations. The cover member can have a variety of outside profiles. For example, the outer surface of the cover member can be smooth, corrugated, have ribs, or other textures. The cover member may be formed of different materials. For example, the cover member may be formed of silicone, parylene, a hydrophilic material, or a lubricious material. In some implementations, the cover member 190 includes antibiotic or an antimicrobial materials or features. For example, in some implementations, the cover member includes rifampin (rifampicin) and/or minocycline HCl. In some implementations, the cover member may include antibiotics impregnated into the material of the cover member. In some implementations, the antibiotics of the cover member are configured to help protect against infection and/or pyrogens during the surgical healing period and beyond.

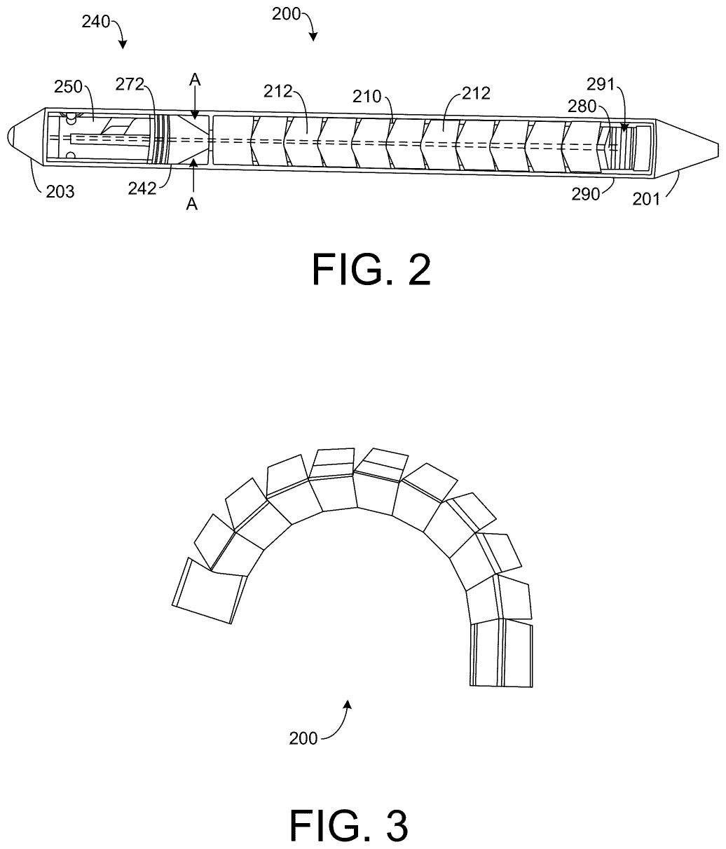

FIGS. 2-22 illustrate an implant or penile prosthesis 200 according to an implementation.

The implant 200 includes an articulating portion 210 and a control portion 240. The implant 200 also includes an elongate member 280 that extends from the control portion 240 to or through the articulating portion 210. In some implementations, the implant 200 may be a penile implant and the implant 200 may be placed within a penis of a patient. Specifically, in some implementations, the implant 200 may be implanted within the corpora cavernosae of the patient. In other implementations, the implant 200 may be placed in other locations within the body of the patient. In the illustrated implementation, the implant 200 includes end portions 201 and 203. While the end portions 201 and 203 are illustrated as being tapered, conical, or frustoconical and have rounded ends, the end portions 201 and 203 may be of any size or shape. Additionally, while the illustrated implementation includes end portions 201 and 203 that include smooth outer surfaces, in some implementations, the end portions 201 and 203 have textured surfaces. Further, while the illustrated implementation includes end portions 201 and 203 that have similar shapes, in other implementations, the end portions have shapes or sizes that are different from each other.

In some implementations, more than one implant 200 may be placed within the body of the patient. For example, in some implementations, two implants 200 may be placed within the body of the patient. Specifically, in some implementations, two implants 200 may be placed within a penis of the patient. The implants may be placed within the penis of the patient in substantially parallel or a side-by-side arrangement. In other implementations, only one implant 200 is disposed or implanted within the body of the patient.

The implant 200 may be placed in a variety of configurations. As will be described in more detail below, the implant 200 may be placed in a curved configuration or a linear or substantially linear configuration. Accordingly, in some implementations, the implant 200 may be placed within a penis of the patient and the patient may selectively place or position the implant 200 in (1) a straight or linear configuration to achieve or simulate an erect penis or (2) a curved or non-linear configuration to achieve or simulate a flaccid penis.

The articulating portion 210 is configured to be placed in a curved configuration and a linear or substantially linear configuration. In some implementations, the linear or substantially linear configuration has less curve or curvature than when the articulating portion is in its curved configuration. Specifically, in the illustrated implementation, the articulating portion 210 (or the implant 200) is configured to be disposed in a linear or substantially linear configuration when the elongate member 280 moves or places locking members of the articulating portion 210 in a first position with respect to articulating members of the articulating portion 210. The articulating portion 210 (or the implant 200) is configured to be disposed in a curved configuration when the elongate member 280 moves or places the locking members of the articulating portion 210 in a second position with respect to the articulating members of the articulating portion 210. The control portion 240 is configured to be manipulated by the user to cause the elongate member 280 to move the locking members from one position to another position.

In the illustrated implementation, the articulating portion 210 includes a plurality of articulating members 212 and a plurality of locking members 214. The articulating members 212 are configured to move, pivot, or rotate with respect to each other. The plurality of articulating members 212 is configured to move with respect to each other such that the articulating portion 210 may be placed in a curved configuration and a linear or substantially linear configuration. In some implementations, the articulating members 212 may move with respect to each other such that the articulating portion 210 may be placed in multiple different curved configurations (for example, on curved configuration that is more curved than a second curved configuration).

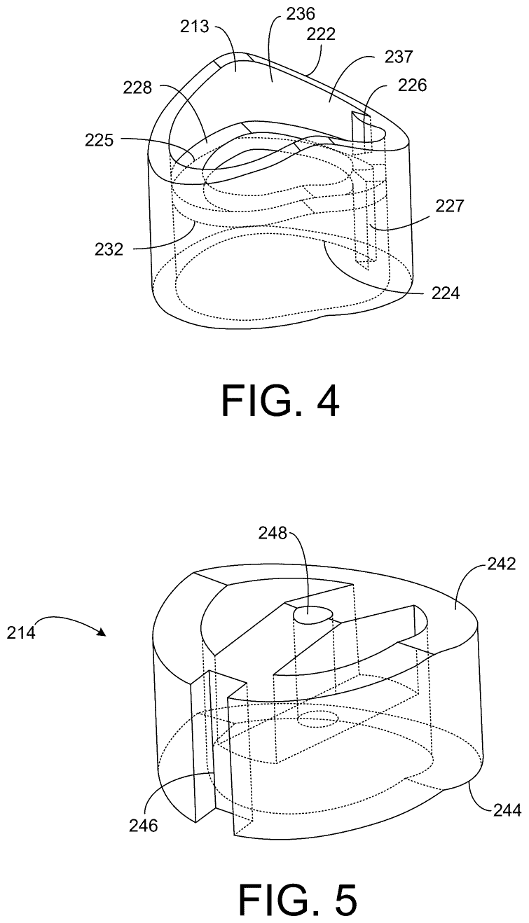

As best illustrated in FIG. 4, the articulating member 212 (each of the plurality of articulating members may have the same or similar features) includes a first end surface 222 and a second end surface 224. The end surfaces 222 and 224 include sloped or angled portions. The first end surface 222 of one of the articulating members 212 is configured to engage a second end surface 224 of an adjacent articulating member. In some implementations, the engagement of the surfaces allows the articulating members to move, pivot, or rotate with respect to each other when the implant 200 is placed in its curved configuration. Although the first end surface 222 and the second end surface 224 are illustrated as having a sloped or curved shape, in other implementations, the first end surface 222 and the second end surface 224 have a different shape or shapes. When multiple implants are placed within the body of the patient, each implant may be configured to have the same curved configurations as the other.

The articulating member 212 also defines a cavity 213 that includes a first cavity portion 236 and a second cavity portion 238. The articulating member 212 includes a first projection 225 that extends into the cavity 213 defined by the articulating member 212 and separates the first cavity portion 236 and the second cavity portion 238. The first projection 225 has a first surface 228 and a second surface 232 opposite the first surface 228. The first cavity portion 236 is configured to receive a portion of a first locking member as will be described in more detail below. The second cavity portion 238 is configured to receive a portion of a second locking member as will be described in more detail below.

The articulating member 212 includes a second projection 226 (or engagement portion) that extends into the cavity 213 defined by the articulating member 212 and a third projection 227 (or engagement portion) that extends into the cavity 213 defined by the articulating member 212. As will be described below, the second projection 226 is configured to engage a locking member to help retain the rotational relationship of the locking member and the articulating member 212 and to allow the locking member to move with respect to the articulating member 212 to allow the articulating portion to be placed in a locked (linear or substantially linear) configuration or an unlocked (curved) configuration. Similarly, the third projection 227 is configured to engage another locking member to help retain the rotational relationship of the locking member and the articulating member 212 and to allow the locking member to move with respect to the articulating member 212.

The cavity 213 defined by the articulating member 212 extends from a first end of the articulating member 212 to a second end of the articulating member 212 to form a through lumen. The cavity 213 is configured to allow the elongate member 280 to pass through the articulating member 212. In other words, a portion of the elongate member 280 may be disposed within the cavity 213 defined by the articulating member 212 and may move within the cavity 213.

The plurality of locking members 214 is configured to fix or lock the articulating members 212 in place with respect to each other. Specifically, the plurality of locking members 214 is configured to engage the articulating members to fix or lock the articulating members in place to place the articulating portion 210 in a linear or substantially linear configuration.

As best illustrated in FIG. 5, the locking members 214 (each of the plurality of locking members may have the same or similar features) includes a first end surface 242 and a second end surface 244. The locking member 214 is configured to be disposed within a first portion of a cavity of a first articulating member and a second portion of a second articulating member. In some implementations, the first end surface 242 of the locking member 214 may be configured to engage a second surface 232 of a first projection 225 of an articulating member 212. Similarly, the second end surface 244 of the locking member 214 may be disposed within a cavity of a different articulating member and the second end surface may engage a first surface of the articulating member.

The locking member 214 defines a groove 246. The groove 246 is configured to receive a projection (such as projection 226) of a first articulating member and receive a projection (such as projection 227) of a second articulating member to help retain a rotational relationship between the locking member 214 and the first articulating member and the second articulating member. Accordingly, in some implementations, the engagement of the projections 226 and 227 and the groove 246 help prevent rotation of the locking member 214 relative to the articulating member 212. Although the projections 226 and 227 and the groove 246 are shown as having a particular shape, in other implementations, the projections and the groove have different shapes. Additionally, in some implementations, the locking member 214 does not define a groove and/or the articulating members do not include projections.

The locking member 214 is configured to slide or move along the projection 226 of a first articulating member and the projection 227 of a second articulating member from a first location to a second location.

The locking member 214 defines a lumen 248. The lumen 248 extends through the locking member 214 from a first end portion of the locking member to a second end portion of the locking member. The lumen 248 is configured to receive a portion of the elongate member 280 such that the elongate member 280 extends through the lumen 248. The elongate member 280 is coupled to the locking member 214.

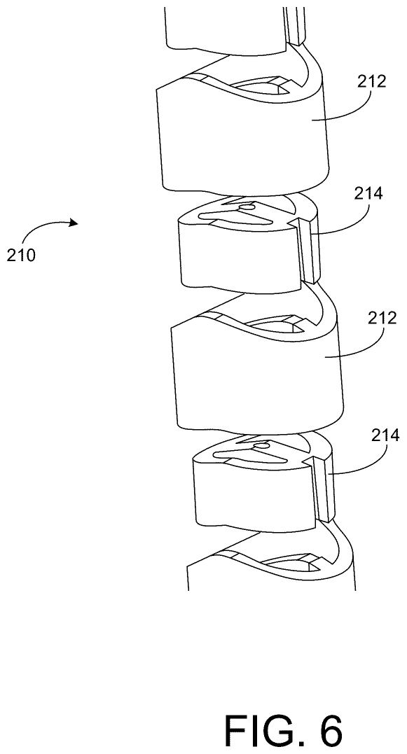

FIG. 6 is an exploded view of a portion of the articulating portion 210. As best illustrated, the articulating portion 210 includes a plurality of articulating members 212 and a plurality of locking members 214. The plurality of articulating members 212 and the plurality of locking members 214 are disposed in an alternating arrangement (each articulating member disposed between a first locking member and a second locking member and each locking member being disposed between a first articulating member and a second articulating member). There can be any number of articulating members 212 and any number of locking members 214. For example, in some implementations there are seven articulating members. In other implementations, there are more than seven articulating members. In yet other implementations, there are less than seven articulating members.

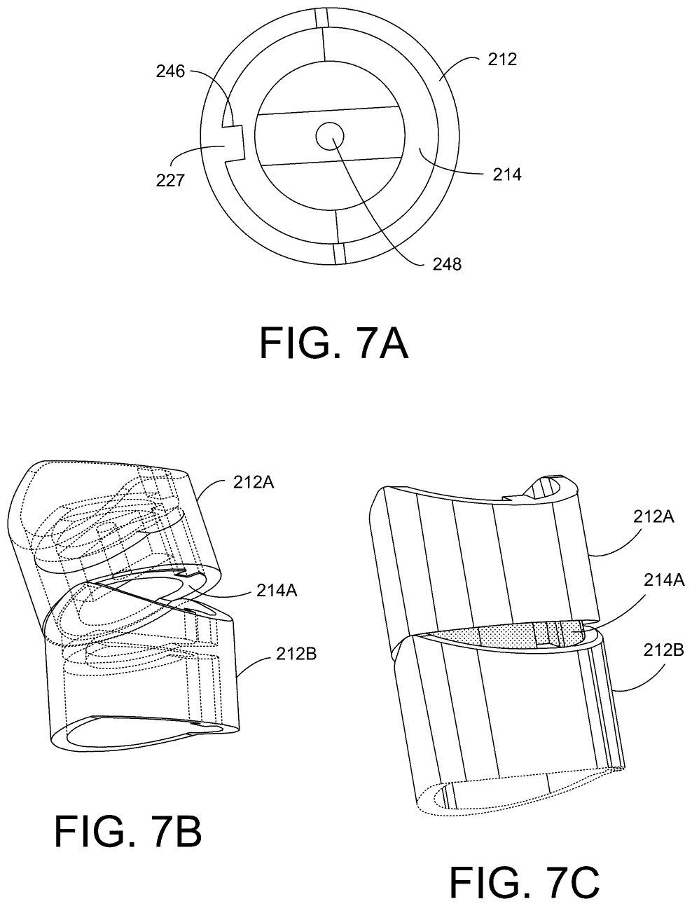

FIG. 7A is an end view of the locking member 214 and the articulating member 212. As best illustrated in FIG. 7, the groove of the locking member engages or receives a projection of the articulating member.

The control portion 240 is configured to be engaged by the user to place the implant 200 (or the articulating portion 210) in a curved configuration or a linear or substantially linear configuration. The control portion 240 includes a plunger member 261, a cam member 250, a housing member 260, a lock button 270, and a biasing member 272. In the illustrated implementation, the user may engage the plunger member 261 (for example at arrow A of FIG. 2) to activate the control portion 240 to move or transition the implant 200 from the curved configuration to the linear or substantially linear configuration. The user may also engage the lock button 270 and the plunger member 261 to move or transition the implant 200 from the linear or substantially linear configuration to the curved configuration.

When the user engages or squeezes the plunger member 261, the plunger member 261 moves against the biasing member 272 and towards the cam member 250. The cam member 250 is moved and rotated to a new position with respect to the housing member 260. An end portion of the elongate member 280 is coupled to the cam member 250. Accordingly, when the cam member 250 is in a first position with respect to the housing member 260 elongate member 280 in a first portion and the locking members are positioned to place the implant in a linear or substantially linear configuration and when the cam member 250 is in a second position with respect to the housing member 260 the elongate member 280 is in a second position and the locking members are positioned to place the implant in a curved configuration. FIG. 7B illustrates a locking member 214A in a first position to place the implant in a linear or substantially linear configuration (locking member 214A is disposed between articulating member 212A and articulating member 212B to lock the position of articulating member 212A with respect to articulating member 212B). FIG. 7C illustrates the locking member 214B in a second position to place the implant in a curved configuration (locking member 214A is disposed within a cavity defined by articulating member 212A such that articulating member 212A may move or rotate with respect to articulating member 212B).

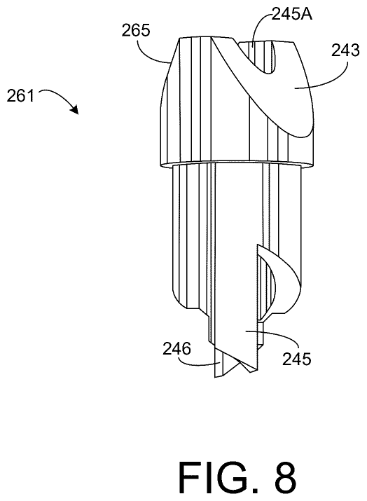

As best illustrated in FIG. 8, in the illustrated implementation, the plunger member 261 includes a first engagement portion 243, a second engagement portion 265, a first extension portion 245, a second extension portion 245A, and a lumen 247 extending through the plunger member 261 from a first end portion of the plunger member 261 to a second end portion of the plunger member 261. The first engagement portion 243 and the second engagement portion 265 are configured to be engaged, such as squeezed by the user, to activate the control portion 240. In other words, the first engagement portion 243 and the second engagement portion 265 may be squeezed by the user to convert the implant 200 from its linear or substantially linear configuration to its curved configuration and to convert the implant 200 from its curved configuration to its linear or substantially linear configuration. In the illustrated implementation, the first engagement portion 243 and the second engagement portion 265 each include angled or slanted surfaces. In other implementations, the engagement portions have different shaped surfaces or surface of a shape different than angled or slanted.

The first extension portion 245 and the second extension portion 246 are configured to contact or engage the cam member 250 when the plunger member 261 is actuated. Accordingly, the extension portions 245 and 245A are configured to contact and move the cam member 250 with respect to the housing member 260. While two extension portions are illustrated, in other implementations, the plunger member includes a different number of extension portions.

The lumen 247 is configured to receive a portion of the elongate member 280. The elongate member 280 is configured to extend through the lumen 247.

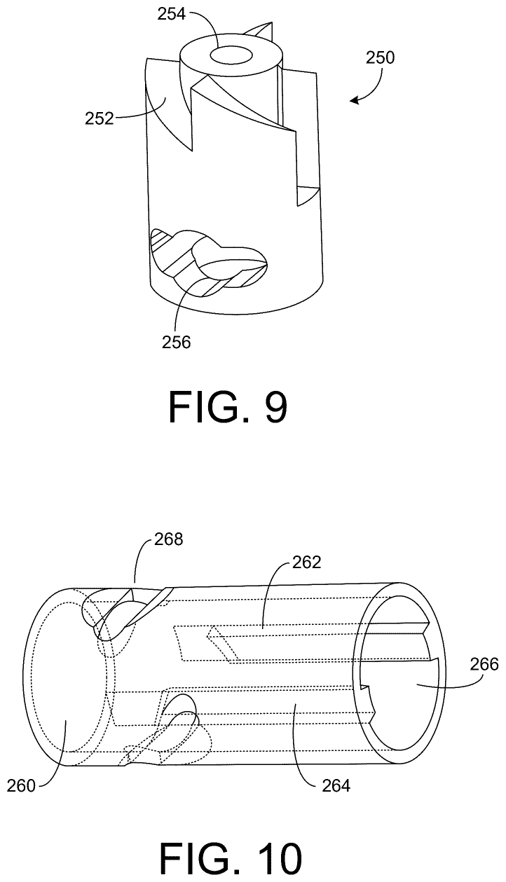

As best illustrated in FIG. 9, the cam member 250 includes an engagement surface 252 and a cavity or lumen 254 and defines an opening 256. In the illustrated implementation, the engagement surface 252 extends around an outer portion of the cam member 250 and includes a series of slanted or angled portions and linear portions. The engagement surface 252 is configured to engage the first extension portion 245 and the second extension portion 245A of the plunger member 261. The engagement with the plunger member 261 causes the cam member 250 to move with respect to the housing member 260. The engagement surface 252 is also configured to engage locking portions 262 and 264 of the housing member 260 to lock the cam member 250 in position with respect to the housing member 260.

The cavity or lumen 254 of the cam member 250 is configured to receive a portion of the elongate member 280. The end portion of the elongate member 280 may be coupled within the cavity or lumen 254 of the cam member 250.

The opening 256 of the cam member is configured to receive the lock button or member 270. The lock button 270 is configured to extend through the opening 256.

As best illustrated in FIG. 10, the housing member 260 defines a cavity or lumen 266. The cavity or lumen 266 is configured to house the plunger member 261 and the cam member 250. The housing member 260 has locking portions 262 and 264 that extend into the cavity or lumen 266. The locking portions 262 and 264 are configured to engage the engagement surface 252 of the cam member 250 to help retain the cam member 250 in place with respect to the housing 260. Specifically, the locking portions 262 and 264 are configured to engage the engagement surface 252 of the cam member 250 until the plunger 261 is actuated by the user.

The housing member 260 also defines an opening 268. The opening 268 is configured to receive a portion of the lock button 270. The lock button 270 is configured to extend through the opening 268 of the housing member 260 when the implant 200 is disposed in the linear or substantially linear configuration. In such configuration, a user may access and engage the lock button 270 through the opening 268. In the illustrated implementation, the housing member 260 defines a first opening 268 and a second opening disposed opposite the first opening 268.

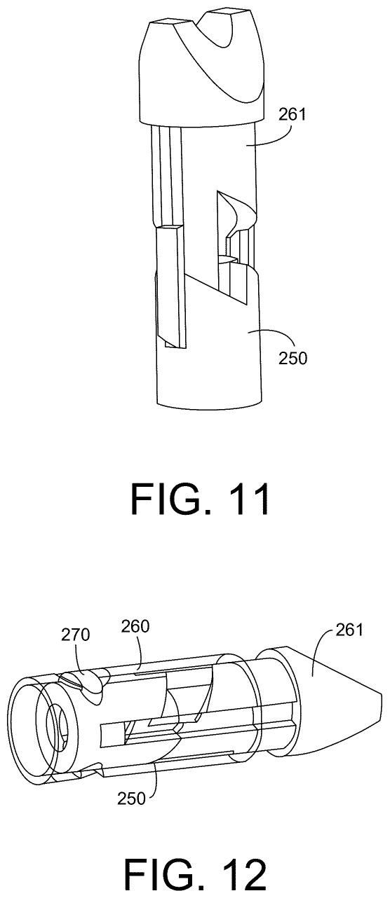

As best illustrated in FIGS. 11 and 12, the plunger member 261 and the cam member 250 are disposed in a collapsed position or configuration. In this position, the elongate member 280 and locking member are in a first position and the implant 200 is in its linear or substantially linear configuration. Also, in this position, the lock button 270 is accessible through the opening 268 defined by the housing member 260.

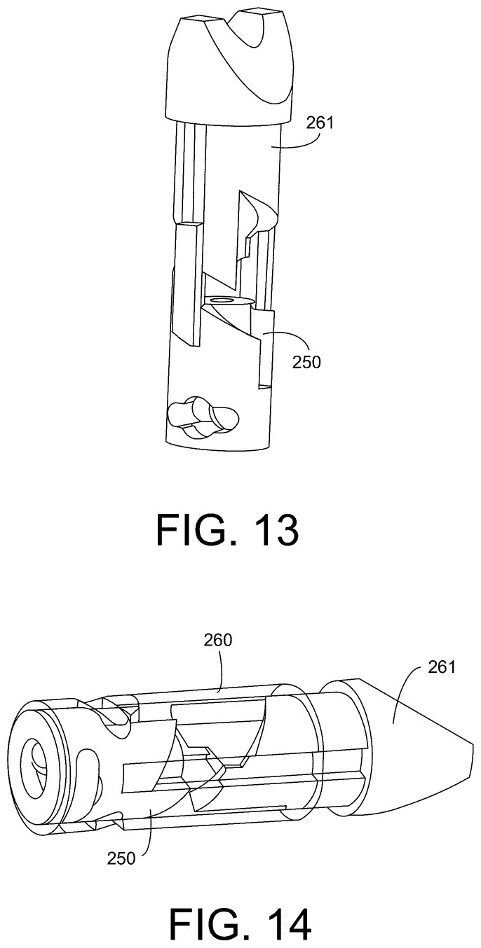

As best illustrated in FIGS. 13 and 14, the plunger member 261 and the cam member 250 are disposed in an extended position or configuration. In this position, the elongate member 280 and the looking member are in a second position and the implant 200 is in its curved configuration.

In the illustrated implementation, the implant 200 includes a bias member 291. The bias member 291 is configured to bias the elongate member 280 to its second position. In the illustrated implementation, the bias member 291 is a spring. In other implementations, the bias member is a different type of bias member.

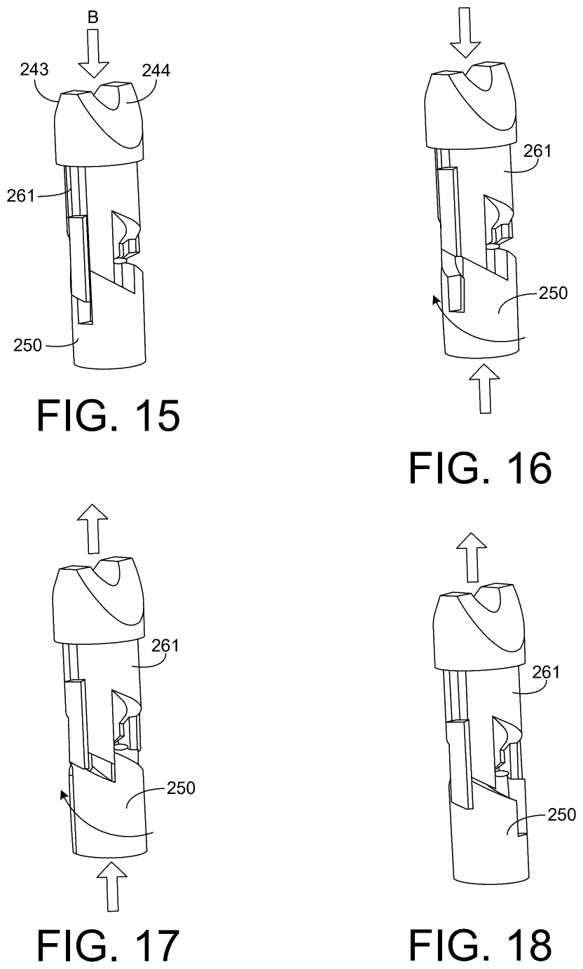

FIGS. 15-18 illustrate the control portion 240 being converted from the position shown in FIGS. 13 and 14 to the position shown in FIGS. 11 and 12 in response to the user engaging the engagement portions 243 and 244 of the plunger member 261. The plunger member 261 moves in the direction of arrow B. The cam member 250 moves and rotates and is locked in place in its new position by the locking portions 262 and 264 of the housing member 260.

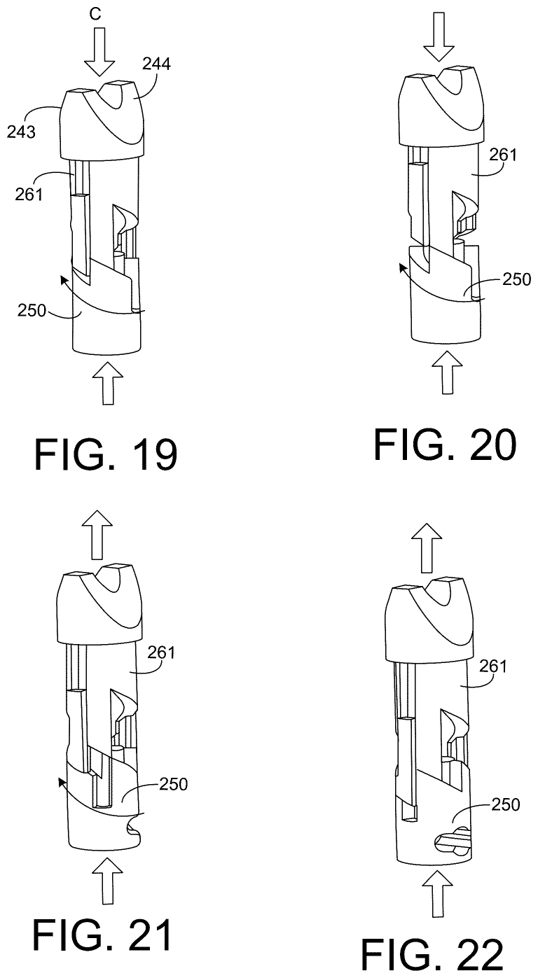

FIGS. 19-22 illustrate the control portion 240 being converted from the position shown in FIGS. 11 and 12 to the position shown in FIGS. 13 and 14 in response to the user engaging the engagement portions 243 and 244 of the plunger member 261. The plunger member 261 moves in the direction of arrow C. The cam member 250 moves and rotates and is locked in place in its new position by the locking portions 262 and 264 of the housing member 260.

In the illustrated implementation, the implant 200 includes a sheath or cover member 290. In some implementations, the cover member extends around or surrounds the outer surface of the implant 200. For example, in some implementations, the cover member defines a cavity and the control portion 240 and the articulating portion 210 are disposed within the cavity defined by the cover member. In some implementations, the cover member is coupled to a portion of the implant 200. In some implementations, the cover member is flexible and/or configured to stretch. In such implementations, the cover member flexes and/or stretches when the implant 200 is moved from one of its configurations to another of its configurations.

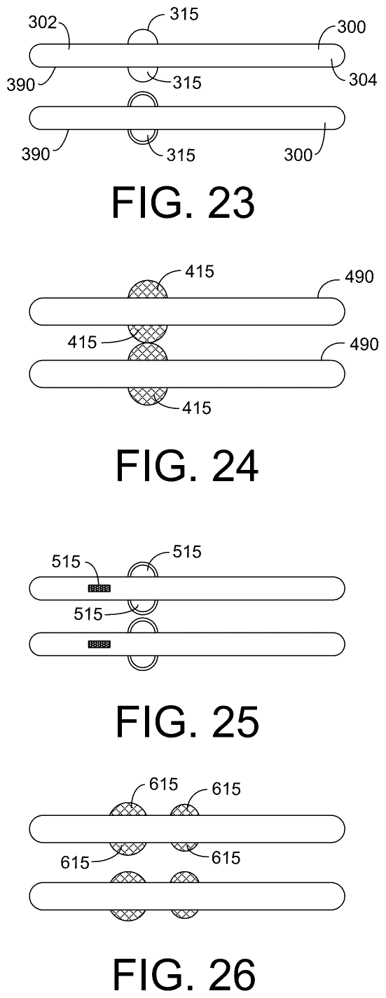

FIGS. 23-26 illustrate implants according to implementations. As illustrated in FIG. 23, two implants 300 may be placed within the body of the patient. For example, the implants 300 may be placed side by side within the body. In some implementations, the implants 300 may be placed side by side within a penis of the patient (such as within the corpora cavernosae of the patient). In the illustrated implementation, the implants 300 includes tabs 315. The tabs 315 are disposed between the proximal end 302 of the implant 300 and the distal end 304 of the implant 300. In some implementations, the proximal end 302 of the implant 300 is placed within the body such that it is closer to the pelvis of the patient than the distal end portion 304 is. In the illustrated implementation, the tabs 315 are disposed closer to the proximal end portion 302 than the distal end portion 304.

In the illustrated implementation, the tabs 315 are configured to facilitate a coupling of the implant 300 to bodily tissue. For example, the tabs 315 may be used to suture the implant 300 in place within the body of the patient to help prevent movement or rotation of the implant 300 within the body of the patient. In the illustrated implementation, the tabs 315 form loops and may be formed with or otherwise extend from the cover member 390 of the implant 300. In the illustrated implementation, the implants 300 each include two tabs 315. In other implementations, the implants 300 include more than two tabs or less than two tabs.

As best illustrated in FIG. 24, in some implementations, the tabs 415 are formed of a solid piece of material. In some implementations, the tabs 415 may be formed of the same material as the cover member 490 of the implant. For example, in some implementations, the tabs 415 may be molded with or formed monolithically or unitarily with the cover member of the implant.

As best illustrated in FIG. 25, in some implementations, each implant includes three tabs 515. The tabs 515 may be disposed offset from each other along the length of the implant and may be offset from each other around the circumference of the implant.

As best illustrated in FIG. 26, in some implementations, each implant includes four tabs 615. The tabs 615 may be disposed offset from each other along the length of the implant.

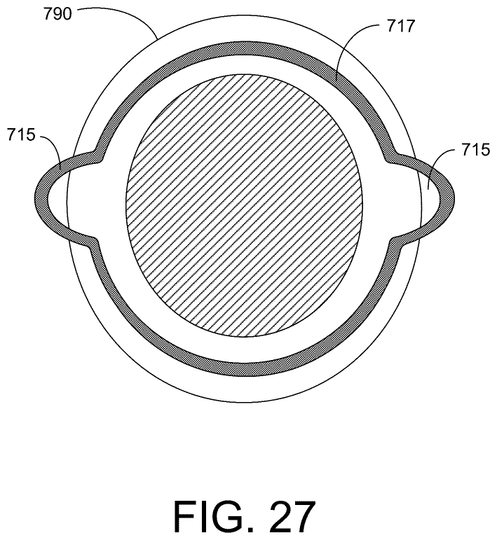

As best illustrated in FIG. 27, in some implementations, the tabs 715 of the implant include a reinforcement member 717. The reinforcement member 717 may be imbedded or otherwise coupled to the cover member 790 of the implant. In some implementations, the reinforcement member 717 is configured to provide strength to the tabs 715 to help prevent ripping or tearing of the tabs 715.

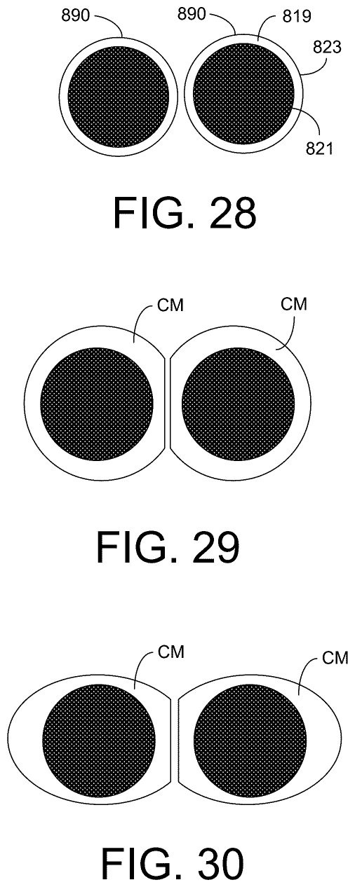

In some implementations, as best illustrated in FIG. 28, the sidewall or wall 819 of the cover member 890 of the implant or implants when two implants are disposed within the body of the patient may have a uniform thickness. In other words, the distance between the inner diameter 821 of the cover member and the outer diameter 823 is the same all the way around the circumference of the cover member. In other implementations, the sidewall may have a non-uniform thickness. For example, in some implementations, the lower portion of the sidewall may have a thinner thickness than the upper portion. In such implementations, the implant may have a tendency to bend more easily downward when the implant is in its curved configuration. In other implementations, the upper portion of the sidewall may have a thinner thickness than the lower portion. In such implementations, the implant may have a tendency to bend upward more easily.

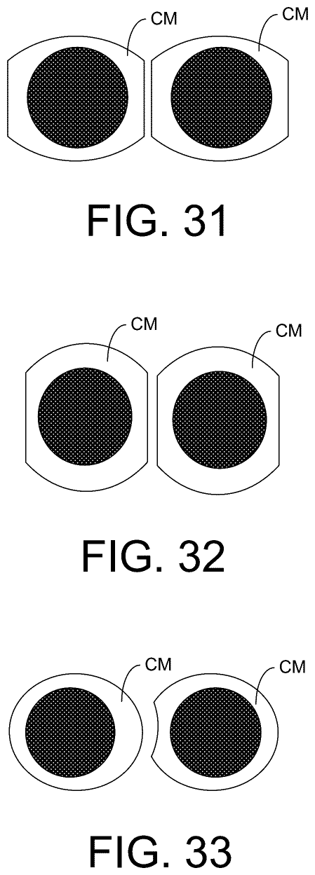

As illustrated in FIGS. 29-34, in some implementations, the outer diameter of the cover member is not circular (non-circular). In some implementations, the outer diameter of the cover member is not circular (non-circular) along the distal portion of the cover member (or distal portion of the implant). In other implementations, the outer diameter of the cover member is not circular (non-circular) along the entire length of the cover member. In some implementations, the non-circular portion of the cover member of one of the implants may engage the non-circular portion of the other implant to help prevent rotation of the implants within the body of the patient.

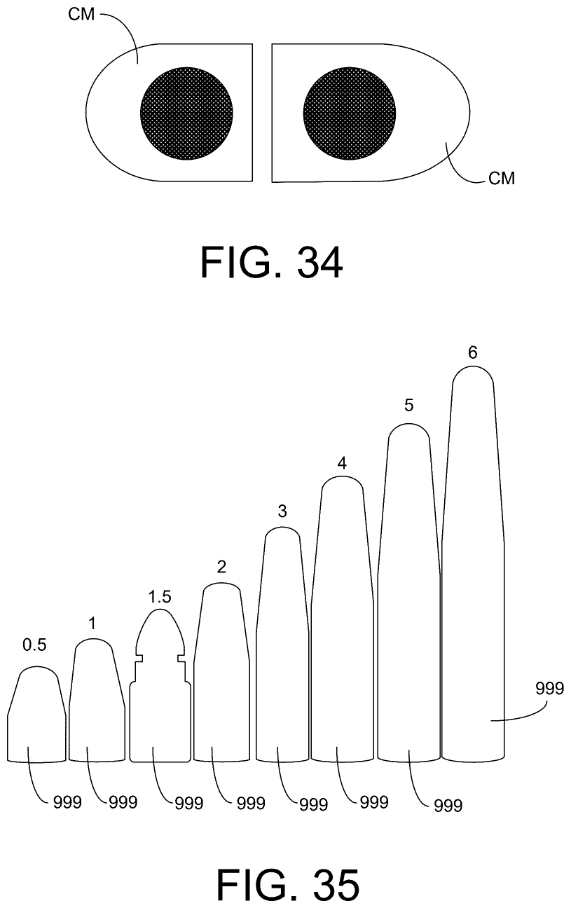

As best illustrated in FIGS. 29, 30, 31, 32, and 34 the cover members CM may have an outer diameter that includes liner or planar portions. The linear or planar portions of one implant may engage the linear or planar portion of the other implant to help prevent the implants from rotating within the body of the patient. As illustrated in FIG. 34, the linear or planar portion may extend across a width of the cover member to allow for as much of a linear portion as possible.

As best illustrated in FIG. 33, one of the implants may have a cover member with an outer diameter that has a concave portion and the other implant may have a cover member with an outer diameter that has a convex portion. The convex portion and the concave portion may interact or otherwise engage one another to help prevent rotation of the implants within the body of the patient.

In other implementations, the cover member may have an outer diameter with a different shape or shapes that allow the cover member of one implant to contact or engage the cover member of another implant to help prevent the implants from rotating within the body of the patient.

FIG. 35 illustrates end extenders 999. The end extenders 999 may be selectively coupled to the ends of the implant. For example, the proximal end or the distal end or both to adjust the overall length of the implant. As illustrated, each of the end extenders 999 has a different length. In some implementations, this allows for selection of the overall length of the implant plus end extenders by the physician to align with the measured intracorporal length of the patient.

Additionally, in some implementations, implant may have an end portion that may be formed of a trimmable material, such as silicone or another trimmable material. Once the implant or the end portion of the implant has been trimmed, an end extender such as those shown in FIG. 35 or another end extender may be coupled to the trimmed end portion of the implant. The interface of the end extender and the implant may include a ridge or step down or the end extender may couple to the implant to form a smooth or flush surface with the end portion of the implant.

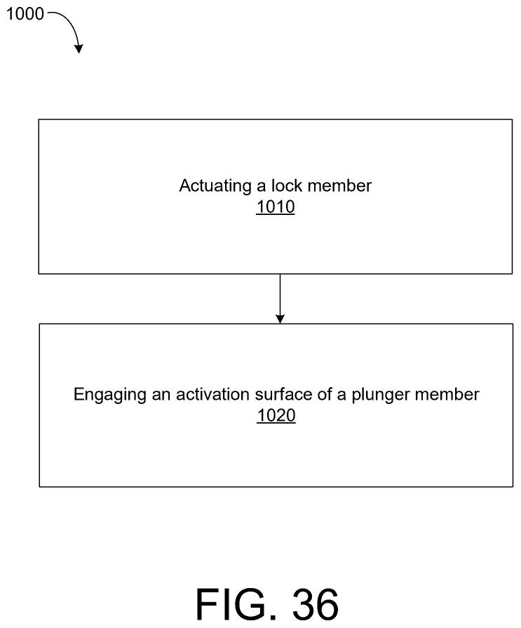

FIG. 36 is a flow chart of a method 1000 of converting an implant according to an embodiment from a linear or substantially linear configuration to a curved configuration. Once the implant is disposed within a body of a patient and is placed in its linear or substantially linear configuration, at 1010 a user may actuate a lock member and at 1020 engage an activation surface of a plunger member of the implant to convert the implant from its linear or substantially linear configuration to its curved configuration.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. A penile implant, comprising:
an articulating portion;
a control portion; and
an elongate member extending from the control portion to the articulating portion.

2. The penile implant of claim 1, wherein the articulating portion includes a plurality of articulating members and a plurality of locking members.

3. The penile implant of claim 1, wherein the articulating portion includes a first articulating member, a second articulating member, and a locking member disposed between the first articulating member and the second articulating member.

4. The penile implant of claim 1, wherein the articulating portion includes a first locking member, a second locking member, and an articulating member disposed between the first locking member and the second locking member.

5. The penile implant of claim 1, wherein the articulating portion includes an articulating member and a locking member, the articulating member defines a cavity, at least a portion of the locking member being configured to be disposed within the cavity defined by the articulating member.

6. The penile implant of claim 1, wherein the articulating portion includes an articulating member and a locking member, the articulating member defines a cavity, at least a portion of the locking member being configured to be disposed within the cavity defined by the articulating member, and the articulating member includes an engagement portion that is configured to engage an engagement portion of the locking member.

7. The penile implant of claim 1, wherein the articulating portion includes an articulating member and a locking member, the articulating member defines a cavity, at least a portion of the locking member being configured to be disposed within the cavity defined by the articulating member, and the articulating member includes a ridge disposed within the cavity.

8. The penile implant of claim 1, wherein the articulating portion includes a first articulating member, a second articulating member, and a locking member, the first articulating member defines a cavity, at least a portion of the locking member being configured to be disposed within the cavity defined by the first articulating member, the first articulating member includes a projection disposed within the cavity, the projection of the first articulating member is configured to be received by a groove of the locking member, the second articulating member defines a cavity, at least a portion of the locking member being configured to be disposed within the cavity defined by the second articulating member, the second articulating member includes a projection disposed within the cavity, and the projection of the second articulating member is configured to be received by a groove of the locking member.

9. The penile implant of claim 1, wherein the control portion includes a plunger member and a cam member.

10. The penile implant of claim 1, wherein the control portion includes a plunger member and a cam member, the plunger member defines a lumen, and the cam member defines a lumen.

11. The penile implant of claim 1, wherein the control portion includes a plunger member, a cam member, and a housing member.

12. The penile implant of claim 1, wherein the control portion includes a plunger member and a cam member, the plunger member having an activation surface.

13. The penile implant of claim 1, wherein the control portion includes a plunger member and a cam member, the plunger member having a first activation surface disposed on a first side portion of the plunger member, the plunger member having a second activation surface disposed on a second side portion of the plunger member, the first side portion being opposite the second side portion of the plunger member.

14. The penile implant of claim 1, wherein the control portion includes a plunger member, a cam member, housing member, and a bias member.

15. The penile implant of claim 1, wherein the control portion includes a housing member and a lock member configured to extend through an opening defined by the housing member.

16. A penile implant, comprising:
a plunger member, a cam member, and an articulating portion having a housing member defining a cavity, at least a portion of the plunger member being disposed within the cavity of the housing member, at least a portion of the cam member being disposed within the cavity of the housing member;
a control portion; and
an elongate member extending from the control portion to the articulating portion.

17. The penile implant of claim 16, wherein the articulating portion includes a plurality of articulating members and a plurality of locking members.

18. The penile implant of claim 16, wherein the articulating portion includes a first articulating member, a second articulating member, and a locking member disposed between the first articulating member and the second articulating member.

19. A method of using a penile implant, comprising:
actuating a lock member; and
engaging an activation surface of a plunger member of the penile implant to convert the penile implant from a linear configuration to a non-linear configuration.

20. The method of claim 19, the activation surface of the plunger member being a first activation surface of the plunger member, the method further comprising:
engaging a second activation surface of the plunger member.
